(19) United States
(12) Patent Application Publication
(10) Pub. No.: US 2021/0212715 A1
(43) Pub. Date: Jul. 15, 2021

(54) WATER JET SURGICAL DEVICE

(71) Applicant: Hydrocision, Inc., North Billerica, MA (US)

(72) Inventors: Paul Kowalski, Haverhill, MA (US); Mark Lewis, Haverhill, MA (US)

(73) Assignee: Hydrocision, Inc., North Billerica, MA (US)

(21) Appl. No.: 17/147,518

(22) Filed: Jan. 13, 2021

Related U.S. Application Data
(60) Provisional application No. 62/960,253, filed on Jan. 13, 2020.

(51) Int. Cl. A61B 17/3203

(52) U.S. Cl. CPC A61B 17/3203; A61B 2017/00323; A61B 2217/005

(57) ABSTRACT

Systems, apparatus and methods for removing tissue of various densities from a surgical site in a minimally invasive manner are provided. The invention may include a fine stream of sterile saline, coupled with a suction effect to cut and remove tissue. A power console may be coupled with a handpiece. The power console may utilize an electric motor, thereby transmitting pressurized sterile saline through a high-pressure tube. The distal end may deliver the saline across a window as a high velocity jet stream which, when coupled with the pressure gradient, pulls and then cuts the target tissue into the cutting window and removes it. The tissue and waste saline may then travel down an evacuation lumen to a waste container. By adjusting pressures, tissue types of various densities may be debrided while minimizing disturbance of surrounding tissue.

Several surgical devices utilize water jets to cut and clear various vessels within the body. However, such devices are not well-suited for all types of vessels. Certain devices, such as those from Hydrocision, Inc. of Massachusetts are capable of pressures as high as 15,000 pounds per square inch (PSI).

Hydrocision utilizes a stream of sterile saline and a simultaneous Venturi suction system to cut and selectively remove tissue of various densities from a surgical site in a minimally invasive manner. While such systems are well-adapted to wound care and spinal markets, peripheral vascular venous and arterial indications, such as deep vein thrombosis and pulmonary embolism, would be desirable. Current treatment options for thrombectomy are limited to anticoagulation therapy, catheter-directed pharmacologic thrombolysis, open surgical thrombectomy, or mechanical or pharm-mechanical thrombectomy. Such treatments are often more invasive, and in the case of anticoagulation therapy, are frequently ineffective at dissolving existing clots. Additionally, current devices are often ineffective at treatment of chronic clots. This results in longer hospital stays and increased risk of major bleeding and related complications.

Moreover, most thrombectomy technologies are poorly adapted from arterial applications. Venous stent occlusions, in particular, form a blockage within a vein. Vein walls are significantly thinner than arterial walls, and are subject to different requirements than arterial walls. More particularly, venous walls contain less smooth muscle and connective tissue, are often of smaller diameter, and are less elastic. The significant differences between arterial and venous systems, and the type of clots that form in each, have resulted in their general ineffectiveness for venous thrombectomy applications. In particular, limited trackability, vessel injury, ineffective treatment for chronic clots, and incomplete revascularization and increased blood loss often result from utilizing arterial systems for venous treatments.

Current systems are generally inadequate at removing adequate volumes of variably aged thrombus, such as acute, sub-acute, or chronic, that often become adhered to venous or arterial vessel walls. As a result, many physicians must resort to using multiple devices, with a variety of mechanisms of action, over multiple sessions, rendering each individual treatment generally inefficient and insufficient. Moreover, this multi-pronged and cost-intensive approach must incorporate catheter-delivered anticoagulants in order to effectively restore vascularization.

It would be desirable, therefore, to provide a differentiated device for removing wall adherent thrombus for acute, sub-acute, and chronic consistencies within a single session.

It would be further desirable to provide systems, apparatus and methods for removing various types of thrombus from a lumen.

It would be further desirable to do so without the need for thrombolytics.

### SUMMARY OF THE INVENTION

Disclosed herein are systems, apparatus and methods for removing tissue of various densities from a surgical site in a minimally invasive manner. The invention may include a fine stream of sterile saline, coupled with a suction effect to cut and remove tissue. A power console may be coupled with a handpiece. The power console may utilize an electric motor, thereby transmitting pressurized sterile saline through a high-pressure tube.

The distal end may deliver the saline across a window as a high velocity jet stream which, when coupled with the pressure gradient, pulls and then cuts the target tissue into the cutting window and removes it. The tissue and waste saline may then travel down an evacuation lumen to a waste container. By adjusting pressures, tissue types of various densities may be debrided while minimizing disturbance of surrounding tissue.

In an embodiment, the invention of the present disclosure may include a catheter and a jet nozzle. The catheter may comprise a distal end, a proximal end, an evacuation lumen, and/or a jet lumen. In an embodiment, the distal end is configured and adapted to remove thrombus, other waste, or other biological material. In a further embodiment, the evacuation lumen and the jet lumen are disposed within the catheter between the distal end and the proximal end. In such a further embodiment, the evacuation lumen and the jet lumen may run parallel to each other.

In an embodiment, the evacuation lumen and/or the jet lumen are in communication with a console. The console may pump liquid from the proximal end of the catheter to the distal end of the catheter. The console may also aid in removal of thrombus or waste from the distal end of the catheter to the proximal end of the catheter.

In an embodiment, the jet nozzle may be located on the distal end of the catheter and may be in liquid communication with the console. The jet nozzle may be configured and/or angled such that the jet stream leaving the jet nozzle is aimed towards the evacuation lumen and/or thrombus. In an embodiment, the jet stream is a stream of saline or saline solution.

In another embodiment, the distal end includes a window. The window may be in the sidewall of the catheter. The window may enable thrombus to partially enter the catheter so that the jet stream may cut the thrombus. In such an embodiment, the thrombus may then be sucked from the distal end to the proximal end via the evacuation lumen.

In an embodiment, the jet nozzle is adjustable. In an alternate embodiment, an end cone is disposed or fastened on the distal end. In an embodiment, the Venturi Effect is employed to move waste from the distal end to the proximal end. In an alternate embodiment, an external source of suction is employed to move the waste from the distal end to the proximal end.

In an embodiment, the invention of the present disclosure further includes a guidewire lumen disposed within the catheter between the distal end and the proximal end. The guidewire lumen may be configured and sized to accept a guidewire. In an alternate embodiment, a guidewire is disposed on the distal end without the need for a guidewire lumen. In a further embodiment, the guidewire may be flexible, allowing for a user to steer the catheter.

In an embodiment, a cage is disposed on the distal end, partially covering the jet nozzle. The cage may comprise at least one strip attached to one of the following: the catheter, the evacuation lumen, or the jet lumen. In a further embodiment, a handpiece may be disposed between the console and the distal end. The handpiece may also be disposed between the console and proximal end or between the distal end and proximal end. In an embodiment, the catheter is configured to be steerable. Thus, the distal end may have an articulated tip.

In an embodiment, the invention of the present disclosure comprises a jet tube. The jet tube may be disposed within the evacuation lumen or the jet lumen. In an embodiment, the jet tube comprises a jet tube distal end and a jet tube proximal end. The jet tube distal end may be bent at an angle (for example, a 90° angle). In such an embodiment, the jet nozzle may be disposed on the underside of the jet tube distal end.

In an embodiment, the jet tube may be configured in a forward cutting design. In such an embodiment, the jet nozzle may be configured to spray a jet stream forward, past the distal end. In another embodiment, the evacuation lumen has an evacuation lumen distal end and an evacuation lumen proximal end. In such an embodiment, the jet nozzle may be placed flush with the mouth of the evacuation lumen distal end. Further, the jet nozzle may face downward, into the evacuation lumen.

### BRIEF DESCRIPTION OF THE DRAWINGS

FIG. 1 illustrates a block diagram of an embodiment of the present invention.

FIGS. 2A-2B illustrate a side-cutting embodiment of the device pulling tissue into the window, in order to cut and evacuate thrombus;

FIG. 2C illustrates a forward-cutting embodiment of the device;

FIG. 3A illustrates a two-lumen embodiment of the device;

FIG. 3B illustrates a three-lumen embodiment of the device;

FIGS. 4A-4B illustrate an embodiment of the device fitted with a guidewire;

FIG. 5A illustrates an embodiment of the device with a jet nozzle spraying a stream of liquid into the evacuation lumen;

FIG. 5B illustrates an embodiment of the device with a cage disposed above the jet nozzle and evacuation lumen;

FIG. 5C illustrates an embodiment of the device with a jet nozzle located near the opening of the evacuation lumen;

FIGS. 6A-6D illustrate an embodiment of a jet tube extension;

FIGS. 7-8 illustrate a filter media that may be disposed within a pump;

FIGS. 9-11 illustrate an embodiment of a jet tube with a bent distal end;

FIGS. 12-14 illustrate an embodiment of a jet tube with a shortened bent distal end;

FIGS. 15-18 illustrate an embodiment of an LC filter configured to accept a jet tube; and

FIGS. 19-21 illustrate an embodiment of a spacer tube configured to be disposed between an LC filter and a jet tube.

While the invention is described with reference to the above drawings, the drawings are intended to be illustrative, and the invention contemplates other embodiments within the spirit of the invention.

### DETAILED DESCRIPTION

The present invention will now be described more fully hereinafter with reference to the accompanying drawings which show, by way of illustration, specific embodiments by which the invention may be practiced. This invention may, however, be embodied in many different forms and should not be construed as limited to the embodiments set forth herein; rather, these embodiments are provided so that this disclosure will be thorough and complete, and will fully convey the scope of the invention to those skilled in the art. Among other things, the present invention may be embodied as devices or methods. Accordingly, the present invention may take the form of an entirely hardware embodiment, an entirely software embodiment, or an embodiment combining software and hardware aspects. The following detailed description is, therefore, not to be taken in a limiting sense.

Throughout the specification and claims, the following terms take the meanings explicitly associated herein, unless the context clearly dictates otherwise. The phrases “in one embodiment,” “in an embodiment,” and the like, as used herein, does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” includes plural references. The meaning of “in” includes “in” and “on.”

It is noted that description herein is not intended as an extensive overview, and as such, concepts may be simplified in the interests of clarity and brevity.

All documents mentioned in this application are hereby incorporated by reference in their entirety. Any process described in this application may be performed in any order and may omit any of the steps in the process. Processes may also be combined with other processes or steps of other processes.

Disclosed herein are devices, systems and methods (the “System”) for treatment of venous thromboembolism (VTE) and related peripheral occlusions, including deep vein thrombosis (DVT) and pulmonary embolism (PE), as well as arterial occlusions.

In certain embodiments the System may be used to treat VTE in place of, or in conjunction with, anticoagulation therapy, catheter-directed pharmacologic thrombolysis, open surgical thrombectomy and mechanical and/or pharm-mechanical thrombectomy. The System may be used in instances where dissolution of existing clots are otherwise unachievable without highly invasive and risky procedures, such as surgical thrombectomy.

The System may be formed of a percutaneous mechanical thrombectomy (PMT) device. The device may include an endovascular catheter. The endovascular catheter may be deployed percutaneously. The percutaneous deployment may be via popliteal or femoral vein access, or other access methods, and may utilize fluoroscopic guidance, or via any suitable approach.

In certain embodiments, the System may be used to treat peripheral occlusions, such arteriovenous fistulas and other arterial needs.

The System may remove tissue of various densities from a surgical site in a minimally invasive manner, using a fine stream of sterile saline, coupled with a suction effect to cut and remove tissue. A power console may be coupled with a handpiece. The power console may utilize an electric motor, thereby transmitting pressurized sterile saline through a high-pressure tube.

The distal end of the tube may deliver the saline across a window as a high velocity jet stream which, when coupled with the pressure gradient, pulls and cuts the target tissue into the cutting window and remove it. The tissue and waste saline may then travel down an evacuation lumen to a waste container. By adjusting pressures, tissue types of various densities may be debrided without disturbing surrounding tissue.

The System may remove wall-adherent thrombus of acute, sub-acute and chronic consistencies within a single session. In some embodiments, the System includes a power console. The power console may utilize common power. The power console may be connected to, and in fluid communication with, a flexible tube. The flexible tube may be a jet tube. The flexible tube may be a catheter, incorporating a jet tube therein. The flexible tube may be flexible. The flexible tube may be formed to deliver sterile saline therethrough.

The tube may include, or be in communication with, a nozzle. The nozzle may be located at the distal-end of the tube. The nozzle may deliver the jet stream downward. For example, the jet stream may be delivered vertically or substantially vertically downward. The jet stream may be delivered, via the nozzle, across a window as a high pressure jet stream. The window may be small, and may be formed in the distal end of the nozzle. In an exemplary embodiment, the window may be 0.065″×0.042″×0.042″ in width, length and depth, respectively. In an exemplary embodiment, the window may be 0.125″×0.233″×0.189″ in width, length and depth, respectively. In another embodiment, any suitable measurement, may be used.

The jet stream may be a fine diameter jet stream. The jet stream may be formed of saline, or any other suitable fluid, such as water.

In some embodiments, the jet stream of saline may create its own suction. For example, the velocity of the saline may cause suction to be formed, as a result of the Venturi effect. In a further example, the pressure gradient of the saline may cause suction to be formed, resulting from the Venturi effect. In the aforementioned embodiments, the Venturi effect results from the reduction in fluid pressure, when fluid flows through a constricted section. Thus, the differential in flow rate may result in the Venturi effect being utilized. More specifically, the Venturi effect may occur as result of the transition from high to low pressure within the juncture of the jet tube nozzle and the evacuation lumen. Thus, the intensity of the suctions may be correlated with the both the velocity of the jet and the pressure differential. It should be noted that, in some embodiments, the System is specifically formed to maximize the benefits of the Venturi effect, in order to increase suction. This results in pulling thrombus down through a tube, and into a cutting window. As a result, the jet and suction may be formed and adjusted to the needs of the application to act simultaneously and/or in conjunction, in order to cut and remove the thrombus safely.

In accordance with an embodiment of the invention, the Venturi effect may be utilized and tailored to cut and evacuate thrombus of various consistencies—acute, subacute, and chronic clots. Increasing or decreasing the velocity of the fluid in the jet tube or the cutting window size, may alter the intensity of the Venturi effect, thus making the device more suited for certain thrombus consistencies. The device thereby enables cutting and evacuating clots of denser consistencies. In effect, the device of the present disclosure may enable faster performance and more complete reduction or removal of thrombi.

The thrombus and waste saline may be removed via a catheter. The catheter may be located in-line, and may contain an evacuation lumen. In such an embodiment, the jet tube may run perpendicular to the evacuation tube. The jet tube may be attached by stitch welds, mounted on the sidewall of the catheter, or run up through the jet tube lumen and mounted internally. The thrombus and waste saline may be removed via the evacuation lumen and into a waste canister.

In accordance with an embodiment, a waterjet-based system is used to treat and safely remove venous stent thrombus and/or occlusions. The devices and methods may be used to treat post-thrombotic syndrome by removing part or all of a clot.

In an embodiment of the invention, the device includes one or more of a catheter, a jet tube wholly enclosed within the catheter, a guidewire port, and an evacuation lumen. The catheter may be single-use. The device may include an integral source of suction, such as a waterjet creating its own Venturi suction or may be coupled to an external source of suction, such as vacuum power.

The device may further include a console junction mechanism. The console junction mechanism may be a pump cartridge. The pump cartridge may contain a piston and a receiving chamber with inlet and outlet valves. In such an embodiment, the console drives a piston that compresses a fluid in a chamber. Further, in such an embodiment, the outlet valve may open to allow for injection of the fluid into a high-pressure tube. In such an embodiment, the fluid then may enter the proximal end of the handpiece. The console junction mechanism may join the tubing of the catheter to the pump cartridge connection on the console. In another embodiment, a handle assembly may connect to the tubing. The handle assembly may then connect, via a conduit, to the console. In an embodiment, the pump cartridge separates contact of the fluid from the console and may be part of a disposable instrument set.

The catheter may utilize an over-the-wire, monorail, or rapid-exchange system, where the guidewire lumen extends proximally only a short distance from the catheter tip and balloon. Thus, the wire may be inserted into the catheter tip, and exits the catheter shortly thereafter, such that only a single lumen is required. An over-the-wire system may be a system where the guidewire runs through the entire length of the catheter. Monorail and rapid-exchange systems are those where the guidewire only interfaces with a short length of the catheter. This provides savings of time compared with advancing a guidewire through the full length of the catheter. That is, a shorter duration reduces contrast needs and radiation exposure, thereby enabling smaller diameter catheters. In certain embodiments, the catheter may be an over the wire system.

Additionally, an injection and/or flush port may be formed at a proximal end of the device, for flushing and injection of thrombolytics. The flexible jet tube may be split off from the cannula and connected within a handle assembly to a high-pressure tube, such as one made from any suitable material, including, but not limited to, ceramics or KEVLAR. The evacuation lumen may be connected to an evacuation hose, which provides for transmission of saline or other agents, and removes the thrombus.

In one exemplary process, a waterjet is used to cut through a clot, thereby providing for easier balloon dilation of the affected area.

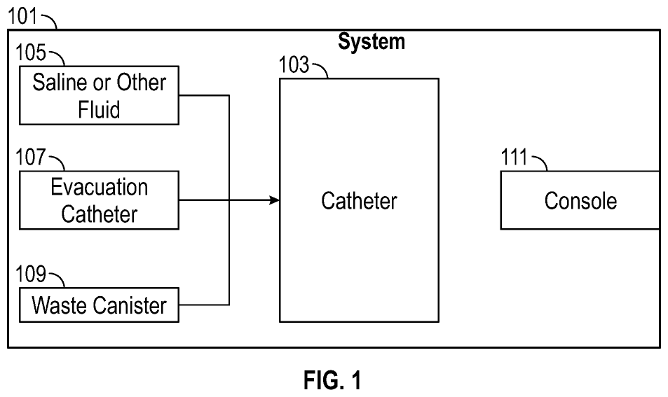

FIG. 1 is a schematic view of an embodiment of the System, illustrated as 101. System 101 may include a catheter 103, a source of saline or other fluid 105, evacuation catheter 107 and waste canister 109. Each of 105, 107 and 109 may be in fluid communication with 103. A console 111 may electro-mechanically control the various components, and may be in communication with a wall-mounted suction device. In some embodiments, the catheter 103 may be connected to a wall-mounted suction device. The console 111 may control various features such as flow rate, pressure, and power regulation.

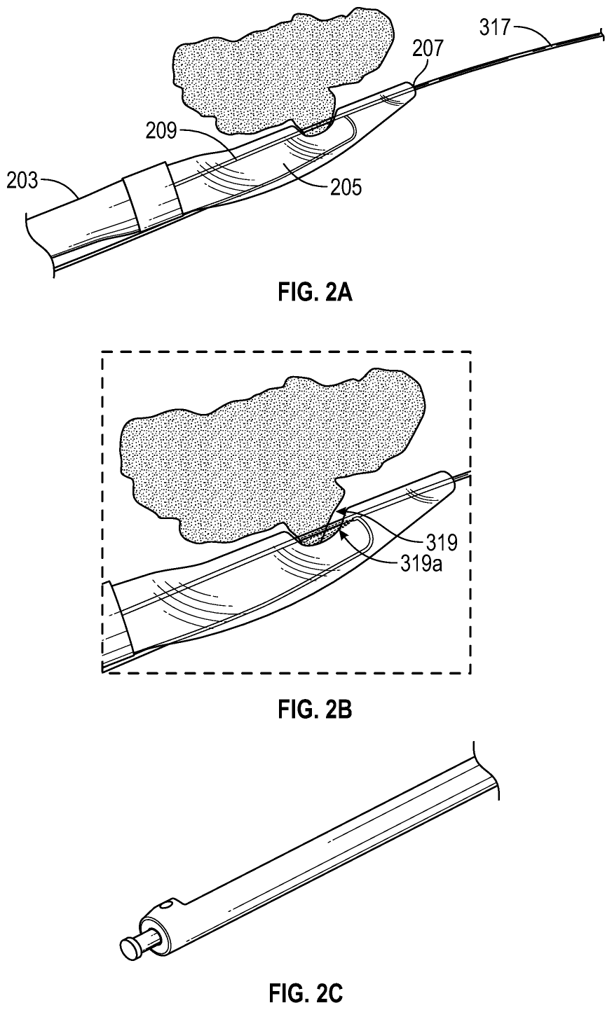

Referring now to FIG. 2A, illustrated is a view of the nozzle/tip 207, attached to catheter body 203. Catheter body 203 may be located distal to the power console 205 (not shown).

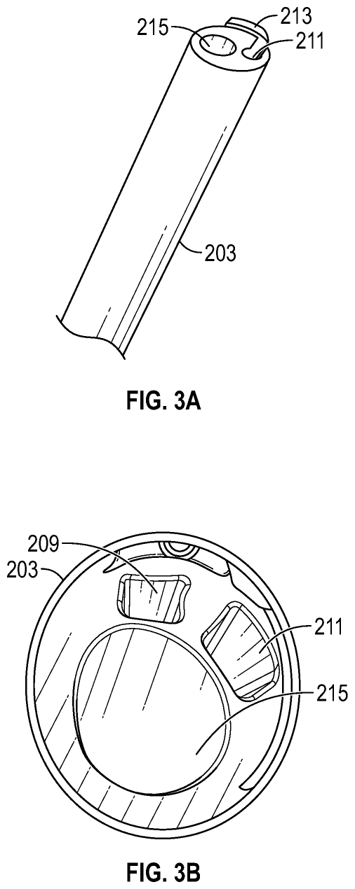

Catheter body 203 may measure between 2 mm (6 French) and 8 mm (22 French) in outer diameter. In certain embodiments, the catheter body may be formed of a diameter of 3.7 mm. In additional embodiments, the catheter body 203 may be approximately 80-170 centimeters in length. In certain embodiments, the catheter body 203 may measure approximately 100 centimeters in length. The catheter may be specifically formed to be less than four millimeters in diameter, allowing it to fit into tighter and harder to reach anatomical cavities. The guide-wire lumen may be optionally formed between 0.014 and 0.035 inches in diameter. Body 203 may include a guide sheath compatible with these measurements. The body 203 may be between approximately 10 cm and 120 cm in length, or any other suitable amount. The catheter body 203 may include guidewire lumen 209. Referring to FIG. 3B, catheter body 203 may further include jet lumen 211.

Guidewire lumen 209 may, in certain embodiments, measure approximately 0.018 inches in diameter, or 0.45 millimeters in diameters. The jet lumen 211 may measure approximately 0.025-0.030 inches in diameter, and an evacuation lumen measuring 0.066 inches in diameter, or 0.055-0.075 inches in diameter. The guidewire lumen 209 may be used for guidewire placement, system guidance, retrieval, or additional tools, or any other suitable form. For example, guidewire lumen 209 may be used for a camera or any other suitable device.

The guidewire lumen 209 may be compatible with various sized guidewires. For example, 0.015 inch and 0.035 inch guidewires may be used. Any other suitably-sized guidewires may be used as well, in accordance with various embodiments.

Referring now to FIG. 3A, jet lumen 211 may incorporate jet tube 213. Jet tube 213 may deliver a water or liquid jet. For example, jet tube 213 may be a water jet based cutting instrument, using saline or any other suitable liquid. The jet tube 213 may receive liquid from a liquid source, such as a wall-mounted spout or in-line liquid source. The liquid may be cycled through the console 111. The console 111 may apply a suitable amount of pressure to the liquid, such as between 2,000-17,000 PSI, thereby causing the liquid to proceed distally down the jet tube 213 instrument, out through the distal tip, and cut thrombus. The jet tube may, for example, consist of a flexible stainless steel material that has a range of 0.016 OD×0.008 ID to 0.025 OD×0.013 ID. The length of the jet tube may be between 8 and 75 inches. Further, the jet tube may be located at a number of positions and angles on the device. In one embodiment, the jet tube may be located on the distal end of the device and may be further positioned to induce various angles of spray. The jet tube may have a cut or hole that acts as a jet tube nozzle. The jet tube nozzle may be cut into the jet tube through use of a laser or an EDM process, 3D printed, or any other suitable process. In another embodiment, the jet tube nozzle may be modular, meaning the jet tube nozzle is manufactured separately and installed in the sidewall of the jet tube. The jet tube 213 may be approximately 0.0025 inches in diameter. In certain embodiments, the jet tube 213 may measure 0.0035-0.0015 inches in diameter.

In certain embodiments, jet tube 213 may be mounted within evacuation lumen 215. This obviates the need for a third lumen, and allows for a smaller diameter catheter. In such embodiments, jet tube 213 may be mounted within, and wholly enclosed within, the evacuation lumen 215. In other embodiments, jet tube 213 may be mounted within a sidewall of the evacuation lumen 215.

Jet lumen 211 may be formed in any suitable diameter. For example, the lumen may be formed with a 0.025 inch outer diameter, or other suitable amounts. In a further example, the lumen may be formed with approximately 0.02-0.05 inch outer diameter, or any other suitable diameter. In yet further examples, increased flexibility may be achieved with a 0.01 outer diameter. Due to the small size of the outer diameter, flexibility is maintained, whereas the tubes being formed of stainless steel allow for pressure containment. In certain embodiments, the lumen may be bent, while maintaining the lumen integrity.

FIG. 3B illustrates an embodiment, with guidewire lumen 209, jet lumen 211, as well as an evacuation lumen 215 located within the catheter body 203. It should be noted that, in accordance with certain embodiments, the measurements of guidewire lumen 209 and/or 211 may be adjusted to accommodate evacuation lumen. Evacuation lumen 215 may be used to evacuate waste resulting from use of the System, including macerated thrombus and used saline.

In another embodiment, a two or three lumen design may be used, with a forward cutting design of the jet tube 213. This is shown in FIG. 3C. The jet tube 213 may extend straight toward the distal end. The jet tube 213 may be extendable. Thus, it may be flush with the distal end of the tip, and extend outward, up to, for example 2.5 mm beyond the tube opening. In another example, the System may incorporate two lumens, an evacuation lumen and guidewire lumen, with the jet tube disposed within the evacuation lumen. The jet tube 213 may incorporate a forward cutting design, with a straight tip, and may extend flush from the distal end of the evacuation lumen and outward, up until a suitable amount, such as 5 mm. It should be noted that a forward cutting design jet tube may be used for chronic clot formation treatment, in particular, for higher-density clots, where a guide-wire cannot extend beyond the clot. Thus, this embodiment may be specifically suited for chronically occluded stents.

Referring back to FIG. 2A, illustrated is tip or nozzle 207, with guidewire 317 extending through and beyond the tip. The guidewire may be placed proximally through the guidewire lumen 209, through the tip 207, and out the distal end of the tip 207. This allows the guidewire to extend beyond the thrombus. This may enable the user to more effectively capture clot material as the catheter is moved distal to proximal. Such a method may be suitable for acute and sub-acute clot consistencies, allowing the catheter to be maneuvered through the clot. It is possible that a chronic clot formation may be too dense, thus the forward cutting design may be needed to work proximal to distal with or without a guidewire. In an embodiment, a user may decide to advance the tip forward facing enough to start, then attempt to advance the rest of the way with the guidewire.

As shown in FIGS. 2A and 3A, jet tube 213 may be formed at an angle. In some embodiments, the jet tube may be formed at a 90 degree, or substantially similar angle. This allows for the jet tube to utilize a jet stream sideways. In another embodiment, the angle may be between 65-125 degrees. Referring back to FIG. 2A, the jet stream may be streamed out the distal end of jet tube 213, toward the side-cutting window 319.

The jet tube 213 may be flexible. Thus, it may create a vertical jet spray in the cut-out window when directed toward the window 219, thereby protecting the jet tube from direct contact with thrombus, but allowing thrombus to enter the window and be cut. In certain embodiments, the thrombus may, after or at the same time as being cut, be simultaneously evacuated.

Tip 207 may be formed with a conical-shaped distal end. This provides for extending the catheter beyond the thrombus, using the guidewire. As shown in FIG. 2A, the distal tip 207b may be angled on one side, such that it can be positioned in close proximity to the wall of the vessel as it is retracted through the thrombus. The angle allows for thrombus to enter the cutting window as it is retracted back through the thrombus. The angle may be between 10 and 20 degrees, thereby allowing for thrombus to be captured along the sidewall of a vessel, as the catheter is pulled back. In another embodiment, the angle may be between 0 and 20 degrees.

It should be noted that the System may be utilized for any suitable treatment or condition, such as venous or arterial clots, acute, sub-acute or chronic clot formations.

Tip 207 may be used for macerating and/or cutting thrombus and other waste. In certain embodiments, tip 207 may be a side cutting instrument. In other embodiments, tip 207 may cut directly forward. In an exemplary process, thrombus, tissue or other waste may be suctioned into the window 319, utilizing the Venturi effect or in-line suction. The thrombus may then be drawn into the window aperture 319a, with the jet stream cutting the thrombus via the jet tube 213. This causes the thrombus to break apart, and is evacuated via a lumen, such as the guidewire lumen or evacuation lumen. It should be noted that removal of thrombus may occur either from direct cutting of the jet spray from the jet tube 213, or from the indirect jet force created from the jet spray, via the Venturi effect, resulting in the Venturi suction. For example, acute clots may require only suction force, created by the jet tube, without direct contact. In accordance with various embodiments, suction force of spray may pull thrombus into the cutting window where the jet then cuts. Thus, the jet and suction act together to macerate tissue and remove it through the evacuation catheter. In other embodiments, the jet may cut first, and tissue is then evacuated through suction effect.

Referring again to FIG. 3B, the three lumens may therefore, in certain embodiments, be formed of: (1) an evacuation lumen (for example, for removal of waste saline and removed thrombus or plaque); (2) a jet lumen (for example, for delivery of high pressure saline and in-line suction); and (3) a guidewire lumen (for example, for guidewire placement and system guidance).

As discussed, in certain embodiments, only two lumens may be used. In some embodiments, the two lumens may be the jet lumen and guidewire lumen. In other embodiments, the two lumens may be the jet lumen and evacuation lumen. In such instances, the guidewire lumen or evacuation lumen may each function dual roles for guidewire and evacuation. In yet other embodiments, only a guidewire lumen and evacuation may be used, with the jet tube mounted within the guidewire lumen or evacuation lumen.

In accordance with certain embodiments, a dual lumen design, such as that shown in FIG. 3A, may be used, with the guidewire lumen functioning as an evacuation lumen as well. The dual lumen design may also be formed to side-cut.

In a further embodiment, a jet tube is mounted inside a two or three lumen catheter. It is fixed in position such that the jet nozzle and spray can be aimed/directed into an evacuation lumen—either in protracted form, and/or contained within the catheter.

The System, in accordance with various embodiments, may be steerable. For example, the steerability may be performed via an angled or articulated tip, or via a shaft. The shaft may be adapted to bend within a guiding catheter. The shaft may be specifically elongated in size, such that it may be delivered from the femoral area (the minimum common femoral vein). The guidewire may also be flexible. In such an embodiment, the flexible guidewire may increase the steerability of the catheter.

In another embodiment, the device may be used to treat a chronically occluded stent, such as a self-expanding stent (for example, a WALLSTENT), resulting in recanalization.

Therefore, in accordance with an embodiment of the invention, the device includes a jet tube, such as a high pressure jet tube. The jet tube may be flexible. In certain embodiments, the jet tube may be formed to withstand a maximum pressure of 17,000 PSI. In an additional embodiment, a third lumen may be used for placement of a 0.014 or 0.035 compatible guidewire (or) use the evacuation lumen for a guidewire. Thus, the flexible jet tube and evacuation tube act as a single flexible endovascular catheter, allowing the structure to be navigable and trackable through a peripheral blood vessel (venous/arterial) while maintaining desired jet tube location and alignment. The design is further formed for sheath compatibility, with a 5-22 French (“F”) measurement.

In one embodiment, the tube is configured to hold a pressure of up to 17,000 pounds per square inch (“PSI”). That is, the jet tube may be formed of stainless steel. Due to the stainless steel, jet tube internal diameter, nozzle properties, pump cartridge properties, and/or console properties, high pressure may be maintained in a safe and efficient manner. In another embodiment, the tube is specifically adapted to hold and operate at a pressure of between 1,500-15,000 PSI. In an embodiment the jet tube may sufficiently remove various clots: level 6-10 is sufficient for chronic clots at 8,000-15,000 PSI with a flow rate of 225 ml/minute; level 3-5 is sufficient for sub-acute clots at 5,500-8,000 PSI with a flow rate of 170 ml/minute; and level 1-4 at 1,500-5,500 PSI with a flow rate of 100 ml/min for acute clots.

Due to the small size and form factor, the flexible jet tube is specifically formed for placement within a catheter. That is, the jet tube is formed to be wholly contained within a catheter, all while maintaining integrity and high pressure flow, and allowing bendability.

In one embodiment, the jet tube nozzle is located on the underside of a bend, in-line with the evacuation lumen. In certain embodiments, the nozzle may be located closer to its shaft, resulting in a smaller catheter diameter. In certain embodiments, the nozzle may be located farther from the shaft, resulting a larger catheter diameter. Thus, in certain indications where a smaller diameter catheter is needed, the nozzle may be located closer to the shaft. In yet additional embodiments, the jet tube may remain partially or completely in the jet lumen. Further, in such an embodiment, the jet tube nozzle may be disposed on the jet tube in a manner that allows the jet tube nozzle to spray fluid at a clot. As a non-limiting example, the jet tube may remain substantially in the jet lumen (or other shaft) and the jet tube nozzle may be steeply angled to effectively spray a clot which the user has positioned along the catheter, but below the distal end. In an alternate embodiment, the jet tube nozzle may be disposed on the jet lumen (or other fluid-carrying shaft) itself. In such an alternate embodiment, the jet tube nozzle may be angled sharply downward (toward the distal end) such that the spray may hit a clot.

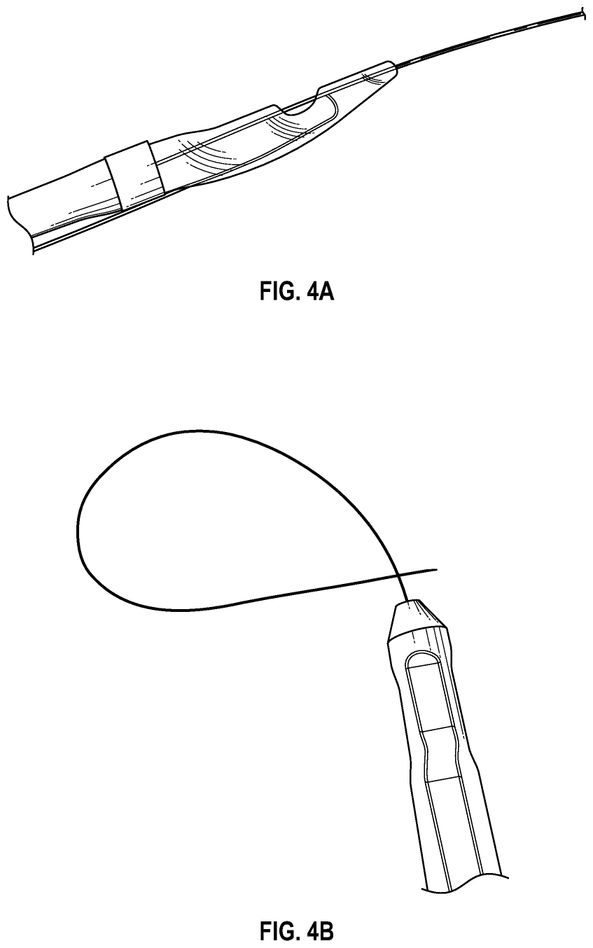

FIGS. 2A-2B and 4A illustrate an embodiment of the catheter where the tip of the distal end includes a sideways facing window. In such an embodiment, thrombus may enter the window and be pelted by the jet spray, effectively cutting part of the thrombus. Further, the thrombus may then be propelled down the evacuation lumen by the suction force created by the Venturi effect or mechanically induced pressure differential. In such embodiments, the jet spray may be directed downward into the evacuation lumen, such that jet spray does not leave the window. Such an embodiment may allow for a high pressure jet spray to be used for cutting while mitigating the risk of the jet spray being directed at the vein's wall and potentially causing damage.

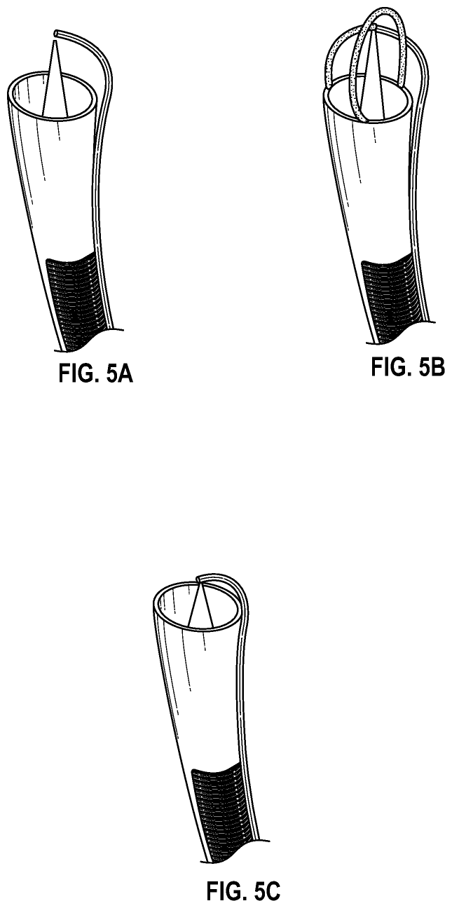

FIG. 5A illustrates one embodiment in which the jet tube, extending to the distal end, includes a jet tube nozzle directed toward the evacuation lumen. In certain embodiments, the jet tube nozzle may spray fluid directly perpendicular to the cross-sectional area of the evacuation lumen. However, in alternate embodiments, the jet tube nozzle may be angled such that the jet tube nozzle's spray incident angle on the cross-sectional area of the evacuation lumen is non-perpendicular.

FIG. 5B illustrates one embodiment of the jet tube located at the distal end, further encapsulated by a cage. In one embodiment, the cage may be formed of metal. In this embodiment, the cage is comprised of at least one strip of metal. In such an embodiment, each end of the at least one metal strip may be attached to portions of the rim of the evacuation lumen, the jet lumen, or other portions of the catheter. In certain embodiments, the cage is formed of at least two strips of metal. In other embodiments, the cage may be formed of any suitable material, such as a polymer or plastic. In an embodiment, the cage may be formed in a height of 1-5 mm, or any suitable amount, and a diameter of 6-22 F. In other embodiments, various other measurements may be used. Such embodiments, as illustrated by FIG. 5B, may protect the jet tube and/or jet tube nozzle, or vessel walls. The cage may protect vessel walls when the jet tube is extended or telescoped to its forward. FIG. 5B illustrates another embodiment of the jet tube and jet tube nozzle. In this embodiment, the jet tube and/or jet tube nozzle is protected by a cage, formed of at least two strips of metal protecting the openness of the tip.

In an alternate embodiment, the distal end of the catheter includes a distal end cone. In such an alternate embodiment, the distal end cone may be configured to enable a user to traverse a thrombus. Further, such an alternate embodiment may include a jet cut into the side of the catheter proximate to the distal end cone. As a non-limiting example, the jet may be disposed under the distal end cone and the jet may be angled with deflection capability. Thus, the cone allows for traversing thrombus and centering guidewire in the vessel.

FIG. 5C illustrated one embodiment where the jet tube is positioned closer to the evacuation lumen. In certain embodiments where a smaller catheter is required, the jet tube may be placed closer to the evacuation lumen. Additionally, in such an embodiment, the jet tube may be adjustable such that a user may decrease the distance between the jet tube nozzle and the evacuation lumen. Due to the contained location, the device may be suitable for acute clots of less dense consistency. In certain embodiments, a distance of, for example, 0 mm-2.5 mm may be used, in order to control the ability to cut clots of differing densities. For example, clots of a first density may be correlated to a distance of 1 mm, whereas clots with a second density may be correlated to a distance of 1.5 mm, or any other suitable amount.

Referring to FIG. 5C, the jet tube nozzle may be positioned on the cusp of the evacuation tube or inside the evacuation tube. In such an embodiment, the device is specifically suited for acute or soft clots that can be acted on by suction alone, without the need for direct contact of the jet with a thrombus. In certain embodiments, the catheter may be angled to achieve certain cutting properties, or may be bendable, with an adjustable angle, to provide a range of cutting abilities. In an additional embodiment, the protected jet tube may be initially deployed in a protected position, and then extended forward vertically by the operator to pre-defined second position, all the while maintaining aim of the jet toward the evacuation lumen. Thus, the jet may come into contact with and remove a more stubborn thrombus. In an additional embodiment, the jet tube or the catheter may be angled to enable side-cutting of a thrombus. In such an embodiment, the jet tube or the catheter may be pliable, allowing a user to adjust the angle, providing for a range of cutting capabilities.

Various embodiments include fixing a jet location vertically within a vessel. The jet location may be fixed such that it is visible in terms of one or both of location and direction to the individual utilizing the device, thereby ensuring adequate treatment and safety. Horizontal movement is then maintained in a locked position, ensuring continual and proper aiming of the jet toward, or inside, the evacuation catheter.

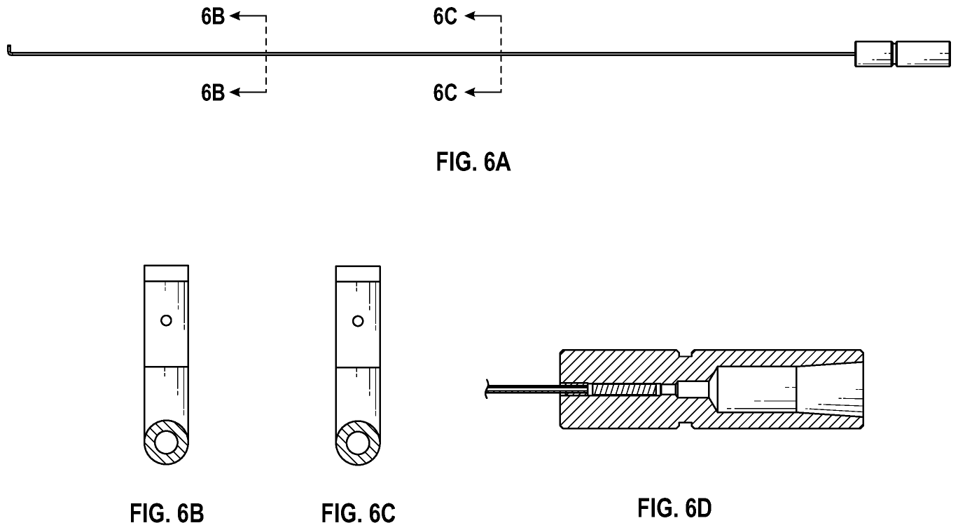

FIGS. 6A-6D are an illustration of an embodiment containing a jet tube extension. In such an embodiment, the jet tube extension may include a jet tube, a spacer tube, an LC filter, and filter media.

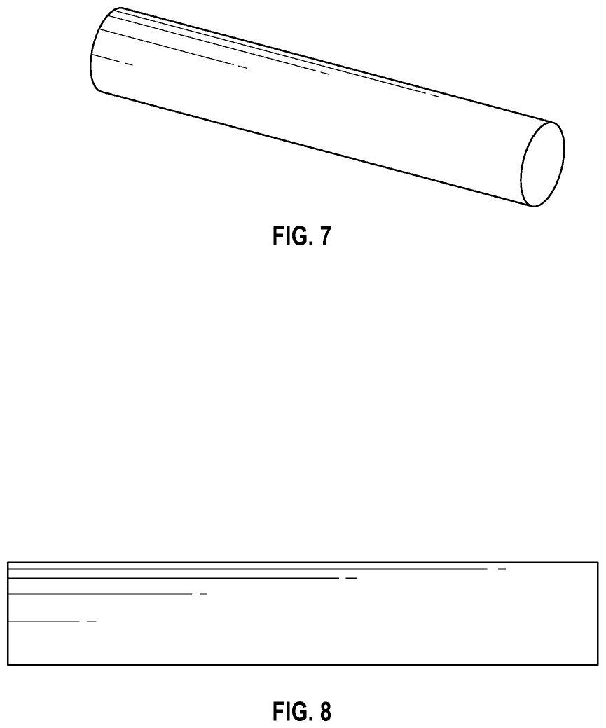

FIGS. 7-8 are an illustration of a filter media that may be disposed within the pump cartridge, the jet tube, the LC filter, or other components of the device. The filter media may be composed of stainless steel braided wire. In other embodiments, the filter media may be made from any material that allows some degree of fluid flow.

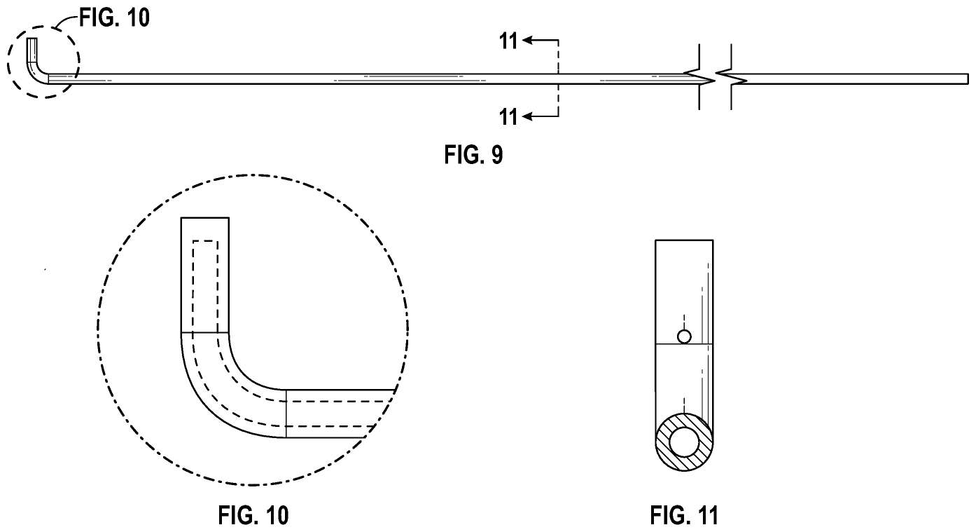

FIGS. 9-11 address an embodiment of a jet tube. As illustrated in FIG. 9, the distal end of the jet tube includes a 90 degree bend. In such an embodiment, the distal end of the jet tube is sealed such that fluid is direct through the jet tube nozzle. The distal end of the jet tube may be made smooth so that it has no sharp edges. The distal end of the jet tube may be bent such that the flat end of the jet tube is 0.115″ from the furthest outer sidewall of the jet tube. FIG. 10 illustrates an embodiment where a jet tube nozzle is drilled into the bottom of the bend of the distal end of the jet tube. In an embodiment, the jet tube nozzle is drilled to a 0.005″ diameter. In another embodiment, the backside of the bend of distal end of the jet tube may be laserwelded if the drill punctured the surface when drilling the jet tube nozzle.

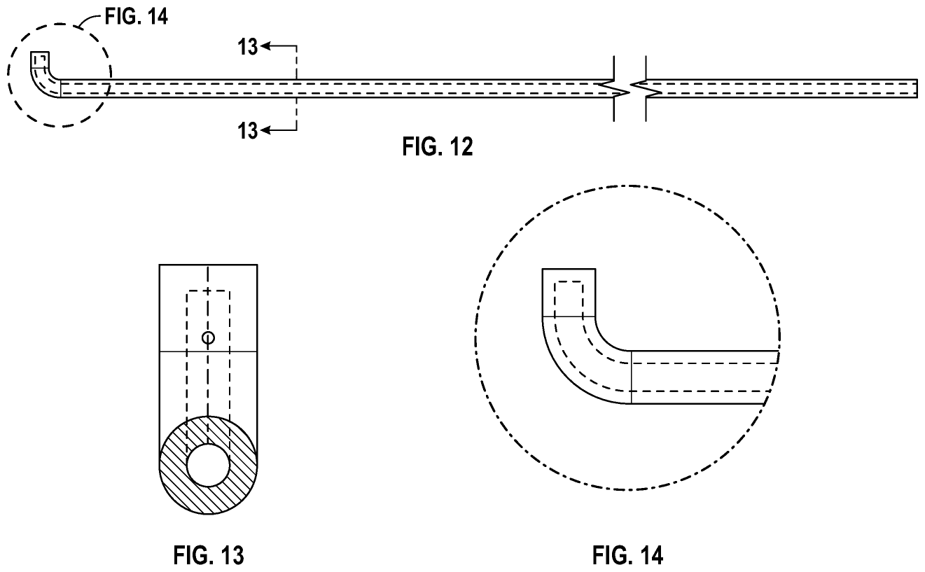

FIGS. 12-14 illustrate further embodiments of a jet tube. In such embodiments, the bent distal end of the jet tube may not jut out as far as other embodiments. In another embodiment, the jet tube nozzle has a diameter of 0.003″.

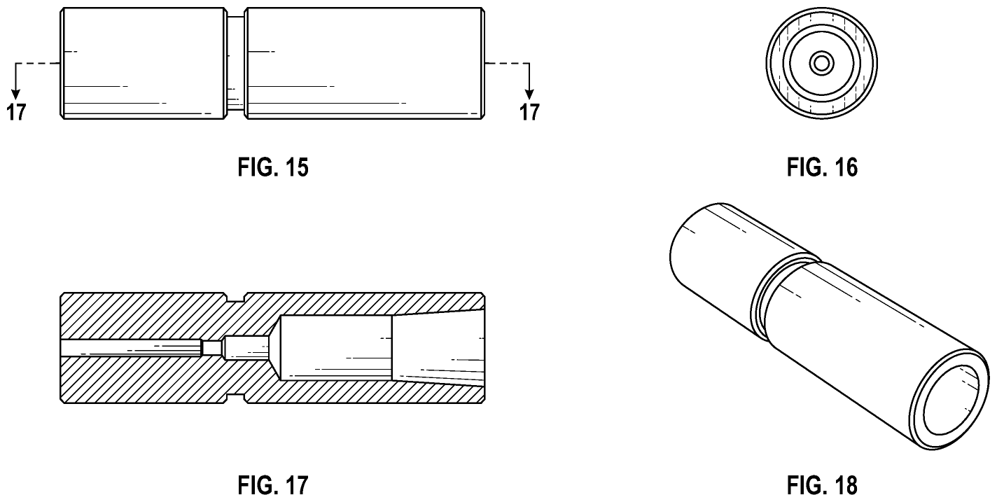

FIGS. 15-18 illustrate an LC filter. In an embodiment, the LC filter may have a distal end and a proximate end. The distal end may be configured to accept the jet tube. The proximate end may be configured to accept an input of fluid. The LC filter may contain a channel that traverses the inside of the LC filter. In an embodiment, the diameter of the channel may decrease as the channel progresses from the proximate end to the distal end. However, in various embodiments, the dimensions of the channel of the LC filter may differ. In an embodiment, filter media may be disposed within the channel.

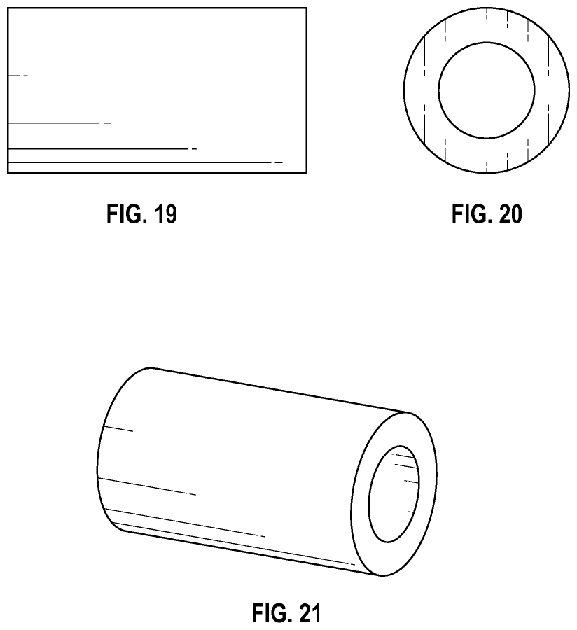

FIGS. 19-21 illustrate a spacer tuber. In an embodiment, the spacer tube is a cylinder with a channel stretching from the distal end of the spacer tube to the spacer tube's proximate end. In an embodiment, the spacer tube is 0.080″ long. In another embodiment, the spacer tube is sized to fit into the distal end of the LC filter. In a further embodiment, the spacer tube's channel is sized to accept the jet tube.

While this invention has been described in conjunction with the embodiments outlined above, many alternatives, modifications and variations will be apparent to those skilled in the art upon reading the foregoing disclosure. Accordingly, the embodiments of the invention, as set forth above, are intended to be illustrative, not limiting. Various changes may be made without departing from the spirit and scope of the invention.

What is claimed is:

1. A surgical instrument comprising:
a catheter comprising:
a distal end configured and adapted to perform a thrombus removal;
a proximal end;
an evacuation lumen disposed within the catheter between the distal end and the proximal end, the evacuation lumen in communication with a console and configured to channel a waste from the distal end to the proximal end; and
a jet lumen disposed within the catheter between the distal end and the proximal end, the jet lumen configured to channel a liquid from the proximal end to the distal end; and
a jet nozzle disposed on the distal end of the catheter, the jet nozzle in liquid communication with the console.

2. The surgical instrument of claim 1, wherein the jet nozzle is configured to spray a liquid jet toward the evacuation lumen and the thrombus.

3. The surgical instrument of claim 1 further comprising a window disposed on the distal end of the catheter, the window configured to allow the thrombus to partially enter the catheter such that the thrombus intersects the liquid jet propelling from the jet nozzle.

4. The surgical instrument of claim 1 further comprising a guidewire projecting from the distal end.

5. The surgical instrument of claim 4 further comprising a guidewire lumen disposed within the catheter between the distal end and the proximal end, the guidewire lumen configured and sized to accept the guidewire.

6. The surgical instrument of claim 4, wherein the guidewire is flexible and configured to allow a user to steer the catheter.

7. The surgical instrument of claim 1 further comprising a cage disposed on the distal end such that the cage partially covers the jet nozzle.

8. The surgical instrument of claim 7 wherein the cage comprises at least one strip attached to one of the following: the catheter, the evacuation lumen, or the jet lumen.

9. The surgical instrument of claim 1, wherein the jet nozzle is adjustable.

10. The surgical instrument of claim 1 further comprising an end cone disposed on the distal end.

11. The surgical instrument of claim 1, wherein a handpiece is disposed between the console and the distal end.

12. The surgical instrument of claim 1, wherein the Venturi Effect is employed to channel the waste from the distal end to the proximal end via the evacuation lumen.

13. The surgical instrument of claim 1, wherein an external source of suction is employed to channel the waste from the distal end to the proximal end via the evacuation lumen.

14. The surgical instrument of claim 1, wherein the liquid is saline.

15. The surgical instrument of claim 1, further comprising a jet tube configured to channel the liquid from the proximal end to the distal end.

16. The surgical instrument of claim 15, wherein the jet tube is disposed within the evacuation lumen.

17. The surgical instrument of claim 15, wherein the jet tube comprises a jet tube distal end and a jet tube proximal end, the jet tube distal end being bent at or near a 90° angle, the jet nozzle disposed on the underside of the jet tube distal end.

18. The surgical instrument of claim 15, wherein the jet tube is configured in a forward cutting design, wherein the jet nozzle is configured such that the liquid jet propels the liquid past the distal end.

19. The surgical instrument of claim 1, wherein the evacuation lumen has an evacuation lumen distal end and an evacuation lumen proximal end, wherein the jet nozzle is disposed flush with the evacuation lumen distal end and downward facing on the evacuation lumen distal end.

20. The surgical instrument of claim 1, wherein the catheter is configured to be steerable, the distal end comprising an articulated tip.
